(19) United States
(12) Patent Application Publication
(10) Pub. No.: US 2006/0141489 A1
(43) Pub. Date: Jun. 29, 2006

(54) Method of statistical genomic analysis

(76) Inventors: David B. Allison, (US); Grier Page, (US)

Correspondence Address: BRADLEY ARANT ROSE & WHITE LLP, 200 CLINTON AVE. WEST, SUITE 900, HUNTSVILLE, AL, 35801, US

(21) Appl. No.: 11/180,360

(22) Filed: Jul. 13, 2005

Related U.S. Application Data
(60) Provisional application No. 60/587,319, filed on Jul. 13, 2004.

(51) Int. Cl. C12Q 1/68; G06F 19/00

(52) U.S. Cl. 435/6; 702/20

(57) ABSTRACT

A method of design and analysis of microarray studies has been developed. The method includes classification of the microarrays; measurement of the reliability and validity of expression measurements; estimation of the effect of an experimental manipulation on gene expression; design of a statistical analysis to determine the distinction between biological significance and statistical significance; and inference of conclusions about effects or associations in populations from information obtained on samples.

### CROSS-REFERENCE TO RELATED APPLICATIONS

This application claims priority from U.S. Provisional Application 60/587,319 entitled “Method of Estimating Necessary Sample Size for Genomic Hypothesis” that was filed on Jul. 13, 2004.

### BACKGROUND OF INVENTION

1. Field of the Invention

The invention relates generally to genetic research. More specifically, the invention relates to a method of design and analysis of microarray studies.

2. Background Art

Genomic research involves the study of the totality of genetic sequences of an organism. A genome is the entire set of chromosomes of an organism. The chromosomes are a series of DNA molecules that each contain a series of many genes. Microarrays are a recently developed technique that allows simultaneous measurement of gene expression on thousands of genes from an organism. Often, the interest is in determining if genetic expression patterns are different for two or more groups of organisms that differ with respect to exposure to some environmental stimuli, genotype, age, or some other characteristic. Various techniques have been proposed to quantify the difference in expression between groups for a single gene. Statistical tests for a difference that rely on parametric assumptions or the central limit theorem may be inappropriate for small samples. Small samples are common in microarray studies.

Genomic scientists often test thousands of hypotheses in a single experiment. One example is a microarray experiment that seeks to determine differential gene expression among experimental groups. Planning such experiments involves a determination of sample size that will allow meaningful interpretations. Due to the many tests being conducted, numerous false positives may occur in microarray experiments, prompting a need to control inferential error rates.

### SUMMARY OF INVENTION

In some aspects, the invention relates to a method for statistical genomic analysis, comprising: designing a study of a microarray chips of genomic material that utilizes multi-stage testing; normalizing a measurement of data received from the study; estimating an experimental error of the measurement of data with an error estimator (θᵢ) for the i^{th} gene is \check{θ}ᵢ=(1−Bᵢ)\hat{θ}ᵢ+Bᵢ\tilde{θ}ᵢ, where Bᵢ=((k−r−2)/(k−r))Sᵢ/(Sᵢ+Â), where k is the number of genes, where r is the number of predictor variables, and where Sᵢ is the variance of the i^{th} gene; and inferring conclusions about the expression of the microarray chips of genomic material from the data received from the study based on the calculation of the probability (Xᵢ) that a difference between groups during an experiment happened by chance, where the probability density function for Xᵢ is

$${f}_{{x}_{i}} ( {x}_{i} ; \underline{λ} , \underline{r} , \underline{s} ) = {λ}_{0} {I}_{( 0 , 1 )} ( {x}_{i} ) + {\sum }_{j = 1}^{v} {λ}_{j} {I}_{( 0 , 1 )} ( {x}_{i} ) \frac{{{x}_{i}^{{r}_{j} - 1} ( 1 - {x}_{i} )}^{{s}_{j} - 1}}{B ( {r}_{j} , {s}_{j} )} ,$$

where v is a mixture of beta distributions with parameters rⱼ and sⱼ, j=1 . . . v, and where B(rⱼ,sⱼ)=∫₀¹u^{r}^{ⱼ}⁻¹(1−u)^{s}^{ⱼ}⁻¹du.

Other aspects and advantages of the invention will be apparent from the following description and the appended claims.

### BRIEF DESCRIPTION OF DRAWINGS

It should be noted that identical features in different drawings are shown with the same reference numeral.

FIG. 1 shows a chart of an Empirical Bayes (EB) estimation of gene-specific effects as compared with an ordinary estimation of gene-specific effects in accordance with one embodiment of the present invention.

FIG. 2 shows a graph that compares the power as a function of the effect size of a single stage design and a two stage design in accordance with one embodiment of the present invention.

FIG. 3 shows a chart that categorizes the significance and effect of genes by quantifying the inferential process in microarray research of a sample size in accordance with one embodiment of the present invention.

FIG. 4 shows a graph of true positive rates across p-values in accordance with one embodiment of the present invention.

### DETAILED DESCRIPTION

The present invention involves novel statistical methods for the design and analysis and interpretation of highly dimensional genomic and proteomic data. The term “highly dimensional” means situations in which the number of variables being measured and considered in a single study is large, often in the thousands. This high dimensionality can involve any combination of thousands of genetic polymorphisms, gene expression levels in a microarray study, protein measurements in a proteomic study, or exposure variables as in the case of nutrient or toxicology studies. Such highly dimensional studies pose challenges, particularly because they are often coupled with small sample sizes. The multiplicity inherent in this type of research is especially acute in the field of biomedical research where the underlying biology is often not characterized by anything near a single common pathway.

The approach to microarray analysis can typically be placed into five categories: Classification; Measurement; Estimation; Design; and Inference. Of these areas, the techniques of classification of microarrays are well known to those of ordinary skill in the art. As such, these techniques of classification should be considered as covered by various embodiments of the present invention.

### Measurement

The quality of any microarray experiment can be no better than the quality of the basic measurements that comprise its data. Consequently, it is important to characterize the reliability and validity of expression measurements and incorporate the information on such measurement properties into experimental designs and inferential procedures. Additionally, steps should be taken to improve the measurement properties of arrays (e.g., via optimal ‘normalization’ procedures), characterize the reliability of a single slide or chip as a quality control check, and estimate gene-specific effect sizes using all available information.

There are many sources of systematic variation in microarray experiments which affect the measured gene expression levels. Normalization is the process for removing such variations. Normalization applies to both oligonucleotide (e.g., Affymetrix) and cDNA type chips. There are at least four different classes of normalization to consider: 1) Within chip/slide; 2) Paired slide normalization for dye swap experiments; 3) Multiple slide normalization; and 4) same gene different type of chip (e.g., CFTR on a lung specific chip vs. CFTR on pancreases chip). In order to normalize, a baseline expression level must be generated. There are at least 3 methods for estimating this baseline parameter: 1) All genes on slide; 2) Housekeeping genes; and 3) control/spiked RNA levels. The present invention will to develop methods for both cDNA and Affymetrix type systems that allow for interactions of random and systematic sources of error.

Empirical Bayes (EB) may be used as models of measured expression levels. To estimate measurement error, consider that in hypothetical repetitions of the experiment, the measured fluorescence signal will fluctuate around some mean value that is itself a property of the cell type, the particular gene, and other factors. These fluctuations are due to multiple sources of variation that arise in producing the measurement, such as variation in mRNA samples preparation, fluorescent tag incorporation, optical noise, and cross hybridization. To account for measurement error explicitly, the method models R and G as independent samples from Gamma distributions with a constant shape parameter, which exhibit constant coefficient of variation, are Gaussian-like, and are supported on the positive line. The EB estimate of differential expression is formed as the ratio of two Gamma variables that follow a common Gamma distribution Gamma(a, v):
EBₖ=(Rₖ+v)/(Gₖ+v);
where v=[((a−1)E(R))/c] is a scale parameter assumed to be equal for both R and G, c is a shape parameter that controls the coefficient of variation (cv) in measurement error, a controls the cv of actual gene expression, and E(R) is the overall average intensity in the R channel. This model is reasonably flexible, skewed right, presents increasing variation with increasing mean, and represents prior uncertainty in actual expression levels. Thus, the method provides more precise estimates of differential gene expression and more accurate assessments of significant changes than standard methods by accounting for differential variability in data and it will evaluate their performance via simulation studies and application to real data.

The EB methodology deals with a single microarray at a time and does not attempt to combine data. Combining information from multiple microarrays may be an effective way to obtain accurate estimates of the contribution of different sources of variation. For example, the expression scale parameter could be partitioned into contributions from different genes, different microarray facilities, different technicians, different chip batches, same gene on different types of chips, different RNA preparations, and different growth conditions. An alternative method is an ANOVA method of analyzing microarray data, which expresses the expected value of log-transformed intensity measurements in terms of contributions from such factors and thus simultaneously corrects for extraneous effects and normalizes the data. Every measurement in a microarray experiment is associated with a particular combination of an array in the experiment. To account for the multiple sources of variation in a microarray experiment, consider the linear model:
log(yᵢⱼₖₗ)=μAᵢ+Dⱼ+Gₖ+Vₗ+AGᵢₖ+GVₖₗ+εᵢⱼₖₗ;
where Yᵢⱼₖₗ is the intensity (fluorescence) measurement, μ is the overall average signal, Aᵢ represents the effect of the i^{th} array, Dⱼ represents the effect of the j^{th} dye, Gₖ represents the effect of the k^{th} gene, Vₗ represents the effect of the l^{th} level of an independent variable (IV) under study, AGᵢₖ represents a combination of array i and gene k (i.e., a particular spot on a particular array), and GVₖₗ represents the interaction between the k^{th} gene and l^{th} level of the IV. Error terms εᵢⱼₖₗ are assumed independent and identically distributed with mean 0. Most of these effects are generally not of interest, but account for extraneous sources of variation. The Aᵢ, Dⱼ, and Vₗ terms effectively normalize the data without preliminary data manipulation. Further, one could model the dye-gene interaction, DGⱼₖ, which would account for certain genes showing higher intensity in one channel, even under non-differential expression. However, the effect can also be controlled by a reverse labeling, counterbalanced design.

The effect of most interest is the interaction between the IV (e.g., tissue types) and genes, GVₖₗ. This term captures departures from the overall averages that are attributable to the specific combination of a level l and a gene k. Nonzero differences in GVₖₗ interactions across varieties for a given gene indicate differential expression. When large numbers of similar quantities are being estimated, the estimates of highest and lowest effects will tend to be too extreme. Also, the measured expression fluctuates around a mean fluorescence value that changes from gene to gene. These issues can be addressed by treating the Gₖ and GVₖₗ terms as random effects in the ANOVA model, which will lead to “shrinkage” estimators for these terms.

Given the forgoing, the EB estimates will be utilized as a dependent variable in an ANOVA model using generalized linear models (GLM) with a Gamma distributed error component. This integrated approach combines the normalization process with the data analysis and has several advantages. First, the normalization is based on a clearly stated set of assumptions that can be evaluated using information in the data. Second, the ANOVA analysis systematically estimates the normalization parameters based on all relevant data and properly accounts for the degrees of freedom used to normalize. This model was designed for experiments in which each gene is spotted only once on each array. However, single microarray output is subject to substantial variability. Thus, genes should ideally be replicated on multiple spots on an array, providing a means to directly assess experimental error variance. As one would expect, replication would lead to more reliable measurements and more precise estimation.

This ANOVA approach is similar to the variance components based generalizability theory approach to assessing measurement. Replication could allow for the estimation of a reliability index similar to a test-retest stability coefficient; however, replication could also be viewed more generally as a within-subjects factor. Hence, the present invention includes a method of deriving variance components from an ANOVA model to compute an internal consistency reliability index. Additionally, this model will provide degrees of freedom that would allow one to assess the importance of additional effects in the model.

Simulation results revealed mean-square error as low as 0.05 times the mean-square error of OLS estimators (i.e., the difference between treatment means). We applied the analysis to an example dataset as a demonstration of the shrinkage of EB estimators and of the reduction in mean square error, i.e., increase in precision, associated with EB estimators in this analysis. The method described here may be applied with computer software. The method has many purposes including evaluating whether and to what extent certain conditions affect gene expression. These conditions or factors can include genotype or environmental manipulation. Such a rich data set offers many possibilities, but also poses many challenges to summarize the volume of available data.

A primary objective of many micro-array experiments is to identify which genes are most likely to be differentially expressed. Secondarily, investigators may be interested in examining the amount of change in gene expression, i.e., estimation of the degree of change in expression. Results in statistics show that when estimates (e.g., estimated differences in gene expression) of many parameters (e.g., true gene expression differences) are examined simultaneously, the estimators have a much larger variance, i.e., range in values, than the true parameters. This is because of the additive property of variances; it could be assumed that the parameters (true unobservable values) are random samples from some distribution, such as the normal. The distribution of parameters thus has a mean and a variance and each parameter is assumed to be a random sample from that distribution in which the variance of the parameters describes the variability among parameter values. Likewise, the estimators of the parameters have errors, i.e., differences between the true parameter value and the estimator, which also can be assumed are random samples from a normal distribution. Hence, each estimator may be thought of as the sum of two random variables, a parameter value and an estimation error. An important result from statistics dealing with normally distributed random variables states that the variance of a sum equals the sum of the variances. In this context, the variance of the estimators equals the variance of the parameters (e.g., true differences in gene expression) plus the variance of the estimation errors. As a result, the estimators have a much larger variance than the true parameters so that estimators of positive differences in gene expression tend to be larger than the true values (the parameters) and estimators of large negative differences in gene expression tend to be more negative than corresponding true differences (assuming differences in expression were centered about zero).

In practice, only the estimators are known; true values are unobservable. Thus, we need to think about the problem in reverse, i.e., to infer from the data to unknown parameters rather than from parameters and errors to the data as in the foregoing discussion. The foregoing discussion emphasized inflation of estimators away from zero relative to the true values; in practice, only the estimators are observed with the knowledge that they are inflated and thus need to be shrunk towards zero (or some grand mean value) so they are closer, on average, to the true parameter values. Such estimators, i.e., shrinkage estimators, have been previously developed in a Bayesian context and in a frequentist context.

A fundamental requirement for application of shrinkage estimators is the assumption that the true parameter values can be modeled as realizations of random variables from some probability distribution. In the context of Bayesian statistical estimation, it is assumed that parameters are exchangeable samples from some distribution, commonly a normal distribution. A set of random variables, say θ_{J}, θ₂ . . . ., θ₁₀ is said to be exchangeable if the parameters are samples from a common joint distribution, p(θ_{y}, θ₂,′″, θₖ), that is invariant to permutation of the indexes, i.e., the order, of the random variables. In practical terms, exchangeability implies symmetry in the prior information available on the parameters. If a parameter, θᵢ″ is chosen and another parameter, θⱼ, is chosen at random the indices i and j convey no information that can distinguish θᵢ and θⱼ so that a priori we have the same expectations about their values. Other information could be used in the model, if it could be formalized into a statistical model, which is perhaps the biggest challenge in model-driven approaches. While the assumption that gene expression differences are exchangeable samples from a normal distribution appears to be a restrictive assumption, it is in fact just the opposite, and it is an assumption of ignorance, i.e., no prior information.

Micro-arrays offer an excellent example of simultaneous estimation of many parameters, where parameters in this case refer to true differences in gene expression. The objective is to obtain an estimator of the ensemble of gene expression differences that on average have the lowest mean-square error. Assuming that errors in gene expression measurement are exchangeable samples from a normal distribution, the classical estimator of the difference between treatment means, the ordinary least squares (OLS) estimator, is known to have the smallest variance (i.e., to be the most precise estimator) among unbiased estimators of the difference in gene expression for a particular gene. However, if we consider the ensemble of parameters (differences in gene expression) being estimated to be an exchangeable set of parameters, we have additional information about them, which can provide better (more precise) estimators. In such a case, we have additional information, namely that true gene expression differences are random samples from a common distribution, such as a normal distribution, with known (or estimable) mean and variance.

By way of example, an experiment is modeled in which gene expression measurements are made on n₁ subjects in group 1 and n₂ subjects in group 2. The treatments can be assumed to be any factor separating the two groups such as age, sex, disease, or drug treatment. For each subject, the tissue(s) of interest is collected, extraction is performed, and a single gene expression measurement is generated for each of k genes. The objective is to estimate the parameters, θᵢ(i=1 . . . k), which are the true differences in gene expression between treatment groups 1 and 2 for genes indexed 1 . . . k. The parameters θᵢ, are defined as:
θᵢ≡μ₁ᵢ−μ₂ᵢ,
where μ₁ᵢ is the true mean gene expression for gene i in treatment group 1 and μ₂ᵢ is the true mean gene expression for gene 1 in treatment group 2. The traditional OLS estimator of θᵢ is given by:

$${D}_{i} = {\overline{X}}_{1 i} - {\overline{X}}_{2 i} , \\ where$$

$${\overline{X}}_{1 i} = 1 {n}_{1} {\sum }_{j = 1}^{{n}_{1}} {X}_{1 ij} , \\ {\overline{X}}_{2 i} = 1 {n}_{2} {\sum }_{j = 1}^{{n}_{2}} {X}_{2 ij} ,$$

and X₁ᵢⱼ is the gene expression measurement for the group h, gene i, and subject j.

The estimated variance Dᵢ is

$${V}_{i} = 1 {n}_{1} ( 1 n - 1 {\sum }_{j = 1}^{n} {( {X}_{1 ij} - {\overline{X}}_{1 i} )}^{2} ) + 1 {n}_{2} ( 1 n - 1 {\sum }_{j = 1}^{n} {( {X}_{2 ij} - {\overline{X}}_{2 i} )}^{2} ) .$$

The OLS estimator is well known to have the smallest variance among all unbiased estimators. Conditional on parameters θᵢ and Vᵢ of the Dᵢ, the distribution of the Dᵢ is
Dᵢ|θᵢ,Vₗ˜N(θᵢ,Vᵢ).

The conditional distribution of differences in gene expression, Dᵢ is conditioned on fixed values of the true difference in gene expression, θᵢ, and the observation error variance for gene i, Vᵢ, which in reality are not known. If the ensemble of parameters of interest, namely, θᵢ, for i=1 . . . k, were considered an exchangeable set of random variables, estimators with lower mean square errors are obtainable, which has been shown in a Bayesian context and in a frequentist context. In the hierarchical model, parameters of interest were modeled as exchangeable samples from the normal distribution, meaning that the set of random variables represents a set of similar quantities. In the case of gene expression micro-array data, each θᵢ, represents the true (unobservable) difference in gene expression between two treatment groups for one gene. The θᵢ, are all similar in that they all represent differences in mRNA levels corresponding to a specific gene.

A set of θᵢ, that might not be considered exchangeable would be three parameters representing differences in levels of preprocessed RNA, mRNA, and protein (i.e., gene product), respectively, corresponding to a particular gene. While the three parameters are associated with a single gene, they are unknown quantities representing three fundamentally different biological processes, so we might not think of them as exchangeable quantities. Assuming the parameters e, are exchangeable, we can model them as independent and identically distributed (iid) samples from some distribution, such as a normal distribution. In the classical model, they are assumed to be iid samples from the normal distribution, i.e.,
θᵢ|μ,A˜N(μ,A), i=1, . . . k.

An ensemble of estimators is obtained by application of Bayes law to obtain estimators of the θᵢ, conditional on the data The data consist of the OLS estimators Dᵢ, and known parameters Vᵢ, μ and A, where
μᵢ|Dᵢ,Vᵢ,μ,A˜N(θᵢ*,Vᵢ(1−Bᵢ)),
θᵢ*=(1−Bᵢ)Dᵢ+Bᵢμ,
and
\hat{B}ᵢ=k−3k1Vᵢ(Vᵢ+A).

These equations are simplified where we have given the estimated form of the Bayesian shrinkage coefficient, Bᵢ. EB estimates of the θᵢ, were obtained by using point estimates of Dᵢ, Vᵢ, μ and A estimated directly from the data. Estimation of model parameters followed by substitution of the estimates as known values makes the analysis “empirical” Bayes as opposed to “Bayesian.”

$$\hat{μ} = {\sum }_{i = 1}^{k} {W}_{i} {D}_{i} {\sum }_{i = 1}^{k} {W}_{i}$$

$${W}_{i} = 1 {W}_{i} + \hat{A}$$

$$and$$

$$\hat{A} = {\sum }_{i} {W}_{i} \{ ( kk - 1 ) {( {D}_{i} - \hat{μ} )}^{2} - {V}_{i} \} {\sum }_{i} {W}_{i} .$$

The EB estimators, θᵢ* for θᵢ are obtained by substitution of μ′ and A′ for μ and A in the previous equations. The EB estimator θᵢ* in is a compromise estimator between the prior estimate of θᵢ, μ′,(estimated from the data), and the data, represented by the OLS estimator, Dᵢ. The equation can be rearranged to accentuate the shrinkage property of this estimator as θᵢ*=Dᵢ−Bᵢ′(Dᵢ,−μ′), where the term-Bᵢ′(Dᵢ, −μ′) is the “shrinkage”, i.e., the amount by which the OLS estimator is “shrunk” or “regressed” towards the mean, μ′.

A question often asked is “How do I know how good is the measurement on this particular slide or chip?” To address this issue, note that all spots on the microarray are commonly assumed to be homogeneous experimental units. Hence, measurements should form a random pattern across the chip's or gel's geography. When measurement artifacts occur, they may create patterns of hybridization dependent on geographic position. An extreme example would be the case of a technician inadvertently placing a thumb on an array. When no such artifacts exist, there will be local spikes of information, but overall the intensity level should show no pattern across the microarray's geography. The background hybridization signal should also be uniform across the microarray and should be less liable to the effects of individual genes. Thus, if we compute an index of the extent to which geographic position on a specific microarray's surface predicts signal intensity, then this index may tell us about the reliability of the measurements produced by the chip with lower values of the ‘geography index’ indicating better reliability.

Two broad approaches can be taken to constructing such an index. First, one could regress intensity measurements on linear or non-linear functions of the microarray's Cartesian coordinates. Alternatively, for an array of k genes, we can calculate the kXk matrix of Euclidian distances among the genes/pixels/probes with respect to physical position on the array and then the kXk matrix of Euclidian distances among the genes/pixels/probes with respect to expression intensity. The corresponding elements from these two matrices can then be correlated and the resulting correlation coefficient or ‘geography index’ taken as a putative index of unreliability. The geography indices will be correlated with the average within-case inter-chip measurement discrepancy, thereby validating them as measures of the (lack of) quality for a given array. The geography index offering the best prediction of measurement quality will be installed in cores B, C and D as an ongoing quality control check to allow investigators to determine whether a particular array (or part of an array) has sufficient quality to be retained.

### Estimation

In addition to measuring the amount of expression for a single gene on a single case, there is a need to measure the effect of an experimental manipulation on gene expression. This is a problem in estimation. Though very close to the issue of inference, it is not simply trying to say whether there is an effect, but also to say how big any such effect is. EB is a name for a collection of methods and approaches that are closely related to shrinkage estimation, hierarchical Bayesian methods, random effects models, and measurement error models. EB is not the same as fully Bayesian analysis. The enormous number of different genes analyzed simultaneously in microarray studies is challenging in that if one chooses genes for which evidence of up- or down-regulation looks strongest from among a large number of genes, then effect estimates, such as fold-change values, will be highly biased. However, the large number of things being estimated presents an ideal situation for EB estimation.

The concept behind EB is that there are often two estimates of a population parameter: an estimate based on some prior expectation and a sample estimator from a particular observed data set. Because both the prior model-based estimate and the sample statistic are associated with some error, the best overall estimate is a weighted average of the two available estimates where the weights are inversely related to the individual estimates' variances. The EB estimator has smaller mean square error (MSE) than ML estimators as long as the number of parameters being estimated is ≧3. EB methods are hybrids of classical frequentist methods and fully Bayesian methods because the data are used to estimate all parameters but a ‘prior’ distribution is postulated to describe the parameters' distribution.

The specific EB approach of the present invention is tailored for microarray analysis. Let there be k genes. For each gene there is a parameter θ that describes the effect of the independent variable under study (e.g., ripeness of strawberries) on gene expression levels. Many different metrics could be chosen forθ. We favor the standardized difference between means, i.e., the difference between group means divided by the pooled within-group standard deviation. For example, consider that comparison of gene expression levels in two groups (e.g., ripe vs. unripe). For the i^{th} gene, derive an ordinary sample estimate of θᵢ denoted \hat{θ}ᵢ and an estimate of its variance Sᵢ. Also, calculate a predicted value of θᵢ denoted \tilde{θ}ᵢ via a regression equation in which various gene characteristics as predictor variables are used. These characteristics could be as simple as the mean value of \tilde{θ}ᵢ for all k genes (i.e., a constant), or more complex such as information about sequence, membership in known gene families, or expression effects observed in other studies, species, etc. Finally, the values of \tilde{θ}ᵢ will also have an estimated variance, denoted Â. Given these quantities, an EB estimator of θᵢ can be written as:
\check{θ}ᵢ=(1−Bᵢ)\hat{θ}ᵢ+Bᵢ\tilde{θ}ᵢ;
where Bᵢ=((k−r−2)/(k−r))Sᵢ/(Sᵢ+ [symbol] A) and where r is the number of predictor variables (not including a mean). The EB estimate will lie between the ordinary estimate calculated only on data from one gene in one study and a predicted effect based on all available data.

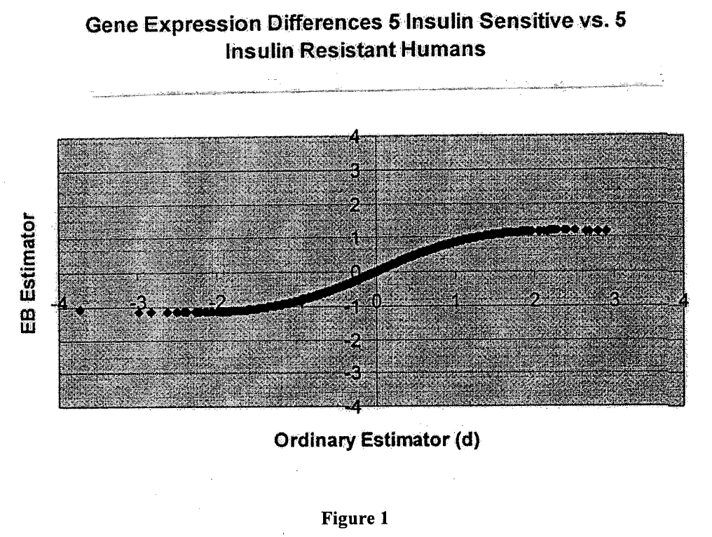

EB estimates of gene-specific effects for microarray data may be calculated with computer programs and used it to analyze real data and conduct simulations. An example relating the EB estimators and the ordinary estimators based on comparing two groups of human subjects (insulin sensitive v. insulin resistant) on over 12,000 genes in FIG. 1. The calculated EB estimates are unbiased and, on average, far closer to the true values than ordinary estimates that only consider information from one gene at a time (i.e., have minimum variance). This is true even when data are not normally distributed, when the case-to-case variance in expression differs greatly across genes, and when the genes are highly correlated but this correlation is ignored. In some difficult simulations, the EB estimator reduced the mean square error (MSE) of estimation by 30% and under the best situations, by nearly 90%.

The EB approach may be extended to allow for correlated gene expression levels across genes. This is conceptually quite simple and can be achieved by calculating the \tilde{θ}ᵢ via generalized least squares regression rather than ordinary least squares regression. However, it involves inverting a kXk matrix and if k (the number of genes) is large, then this may be computationally challenging. Moreover, when k exceeds n (the common case in microarray research) special methods may be needed for rank deficient matrices.

As can be seen in FIG. 1, there is a slight non-monotonicity in the relation between the EB estimator and the ordinary estimator. This effect is slight, dissipates as N increases, and occurs only for very extreme values of the standardized mean difference. It results from the formula for the Vᵢ being a function of \hat{θ}ᵢ in the case of the standardized mean difference.

Finally, the present invention may combine the expression data from each gene its sequence data via computation of an index of sequence-based homology for each pairwise combination of genes. For the i^{th} and j^{th} genes, denote this index S_{i,j}. Next, calculate a predicted value of the standardized mean difference for the i^{th} gene as:

$${\tilde{d}}_{i} ≡ \frac{1}{K} {\sum }_{j = 1}^{k} {S}_{i , j} {\hat{θ}}_{j} for all i \neq j .$$

The vector of \tilde{d}ᵢ can then be used as a predictor in a regression to derive the \tilde{θ}ᵢ. To the extent that sequence is related to expression responses, such regressions should improve prediction of individual gene expression effects.

### Design

The category of design addresses issues related to experimental design of microarray studies. Under the frequentist approach in statistics, the distinction between biological significance and statistical significance must keep in mind. This is due to the fact that if one designs a large enough study, one can find statistically significant differences that have no scientific merit. On the contrary, due to biological system complexity, it stands to reason that very small differences in gene expression could have major biological significance. In such situations it becomes very important to realize that the lack of evidence of an effect does not necessarily indicate a lack of effect, it may simply mean that the study was not powered to detect effects of that magnitude. For this reason, it becomes very important for a researcher to design microarray studies in such a way as to maximize the amount of information obtained.

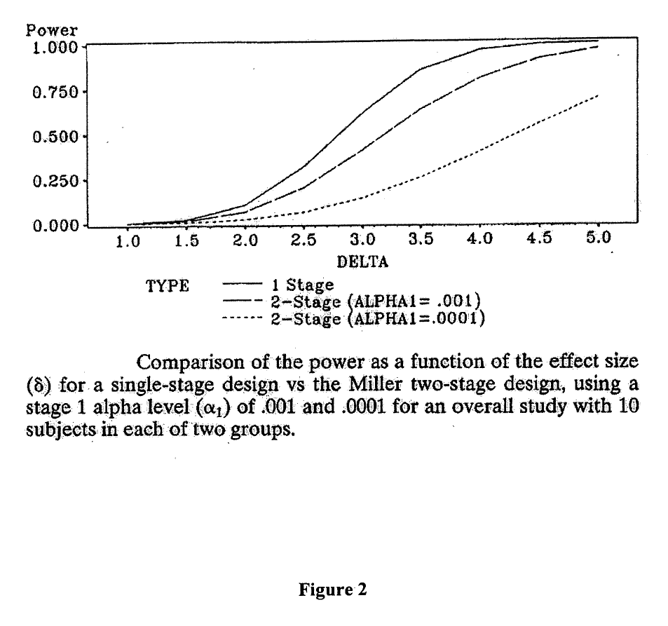

In exploring new designs for microarray studies, the present invention uses multi-stage testing and physical pooling. Two potential advantages of multi-stage testing are: increased power and decreased costs. In the first stage of this method all k genes are tested for differences between two groups using some alpha level greater than that required by a Bonferroni correction. Some number of genes, m (≦k), will have significant effects at this first stage. Then, a second independent set of data is gathered at stage 2 and only those m genes found to be significant at stage 1 are tested at an alpha level of 0.05/m. This method is typically used to increase power (or reduce the type 2 error). However, this method can also decrease power relative to a single stage design as shown in FIG. 2. Furthermore, depending on the choice of alpha level used at the first stage, the two-stage design may lead to an overly conservative experiment wise type 1 error rate. However, the two stage design represents only one possible type of two (or more generally multi) stage design.

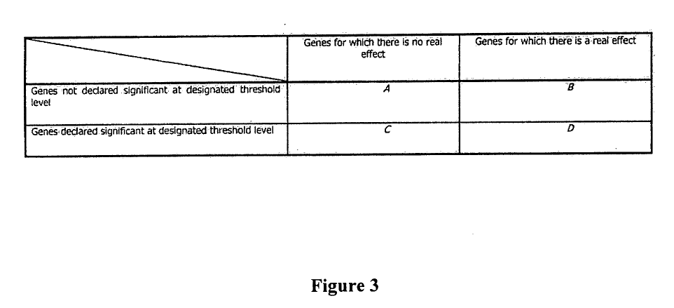

In the planning of a biological experiment, the sample size and power are calculated to be sure to have sufficient samples to address the question being asked. Power is normally calculated for a given effect size. The present invention utilizes a mixture model that allows for the estimation sample size. There are three very important quantities to know when planning a microarray study and these are shown in FIG. 3. The quantities include: the true positive rate

$$TP = \frac{D}{C + D} ;$$

the true negative rate

$$TN = \frac{A}{A + B} ;$$

the false discovery rate and the expected discovery rate or EPD (hereafter referred to as “Zeta”),

$$Zeta = \frac{D}{B + D} .$$

The EPD is the expected proportion of accepted null hypotheses among all true null hypotheses. True positive is the probability that a gene is actually different between the groups when it has been called significant. EPD represents the proportion of genes for which there is a real difference between groups that will be found to be statistical significant, and it is really the average power for all differentially expressed genes rather than the probability to detect a single gene.

Ideally TP, TN, and EPD should be very close to 1 but, in practice, this closeness depends on sample sizes selected for an experiment and different experimenters may be willing to tolerate different departures from these quantities from 1.0 depending on their resources and tastes. It is the interplay between sample size and threshold that can have considerable impact on conclusions that may be inferred from a study's results.

In the present invention, EPD, TP, and TN may be estimated at other sample sizes for any significance level allowing a researcher to choose a sample size that meets ones EPD, TP, and TN requirements. The following technique utilizes a fitted mixture model to a distribution of P-values for which the alternative hypothesis is true and the PDF of the P-values from the study is of the form,

$${f}^{*} ( \underline{p} ) = {\prod }_{i = 1}^{k} [ {λ}_{0} + {λ}_{1} β ( {p}_{i} ; r , s ) ] , 0 \leq {p}_{i} \leq 1 , i = 1 , … , k .$$

Let Z={1,2, . . . ,k}be a set of indices corresponding to the genes in the study, and let T be a subset of Z representing the set of genes that have a true differential expression across two experimental groups, so T⊂Z. The following indicator function is used to distinguish a gene that is truly differentially expressed:

$${I}_{\{ T \}} ( i ) = \{ \begin{matrix}1 & i ∈ T \\ 0 & i ∉ T\end{matrix} for i = 1 , … , k .$$

Thus

$${\sum }_{i = 1}^{k} {I}_{\{ T \}} ( i )$$

represents the true number of genes in a study that are differentially expressed, an unknown quantity in practice since even T is unknown.

Once a statistical test is conducted on the i^{th} gene, a decision is made as to whether to declare that gene as one that is differentially expressed. Typically, this decision is made if the P-value, pᵢ, for the test falls below a predetermined threshold, τ. In the following decision function that, when equal to 1, declares a gene differentially expressed:

$${ψ}_{i} ( {p}_{i} ) = \{ \begin{matrix}1 & {p}_{i} \leq τ \\ 0 & {p}_{i} > τ\end{matrix}$$

where i=1, . . . ,k (hereafter we use the abbreviated notation Ψᵢ). For a specific experiment, the estimates are defined for the values in FIG. 3:

$$\hat{A} = {\sum }_{i = 1}^{k} ( 1 - {ψ}_{i} ) [ 1 - {I}_{\{ T \}} ( i ) ] , \\ \hat{B} = {\sum }_{i = 1}^{k} ( 1 - {ψ}_{i} ) {I}_{\{ T \}} ( i ) , \\ \hat{C} = {\sum }_{i = 1}^{k} {ψ}_{i} [ 1 - {I}_{\{ T \}} ( i ) ] , \\ \hat{D} = {\sum }_{i = 1}^{k} {ψ}_{i} {I}_{\{ T \}} ( i ) .$$

It can be shown that,

E(Â)=A=kλ₀((1−τ), E(\hat{B}) B=kλ₁(1−B(τ;r,s)), E(Ĉ)=C=kλ₀τ,

and E(\hat{D})=D=kλ₁B(τ;r,s) where expectations are taken with respect to the fixed mixture model. So,

$$TP = \frac{D}{C + D}$$

and

$$TN = \frac{A}{A + B} ,$$

defined above, have exactly the same form as TPᵢ and TNᵢ, respectively, except that pᵢ is replaced by the selected threshold τ in the former. EPD has the form,

$$Zeta = \frac{D}{B + D} = \frac{{λ}_{1} B ( τ ; r , s )}{{λ}_{1} ( 1 - B ( τ ; r , s ) ) + {λ}_{0} τ} .$$

TP, TN, and EPD can be estimated using,

$$= \frac{\hat{D}}{\hat{C} + \hat{D}} , = \frac{\hat{A}}{\hat{A} + \hat{B}} ,$$

and

$$= \frac{\hat{D}}{\hat{B} + \hat{D}} ,$$

which are consistent estimators.

The above framework allows an analysis of threshold and sample size considerations for TP, TN, and EPD. Suppose a mixture model has been fitted to a distribution of P-values that were computed using a t-test on each of k genes (this test could be for two independent samples, or paired samples) and is of the form f*(p) shown above.

This procedure can yield estimates of TP, TN and EPD for any given sample size n* and threshold value r. For given values of n* and τ, M independent samples p*=p₁*, . . . , pₖ* are randomly drawn from the mixture model f*(p), with its parameters estimated from the preliminary sample, where M is a sufficiently large number. From these M independent samples of p-values M different estimates [symbol] Pᵢ, [symbol] Nᵢ and [symbol] zetaᵢ are obtained and from these M estimates, Monte Carlo estimate Ê[ [symbol] Pᵢ], Ê[ [symbol] Nᵢ] and E[ [symbol] zetaᵢ] are obtained by averaging the intermediate sample estimates [symbol] Pᵢ;, [symbol] Nᵢ and [symbol] zetaᵢ over the M samples. The estimates [symbol] Pᵢ, [symbol] Nᵢ and [symbol] zetaᵢ are computed from the adjusted p-values p**=p₁**, . . . ,pₖ**, where the p-values p** are obtained from the p-values p* corresponding to the original sample size n, by adjusting these p-values with respect to the new sample size n*. An adjusted p-value pᵢ** is obtained from a p-value pᵢ* by transforming pᵢ* back to the corresponding t-statistic tᵢ* and computing a new p-value pᵢ** using a new degrees of freedom reflecting the new sample size n*.

### Inference

Inference means the process of drawing conclusions about effects or associations in populations from information obtained on samples. The topic of inference is closely related to, but somewhat broader than significance testing and presents several challenges in the microarray context. For example, a major objective in the analysis of microarray data sets is to find those genes that have a significant differential expression between two or more groups or conditions. Finding such genes is challenging because of the large number of genes, k (often larger than 10,000), which is common in microarray data sets. Applying an uncorrected test, for instance the t-test, separately to each of k genes yields an experiment-wise type I error probability (i.e. false positive rate) as high as 1-(1-α)^{k}, when all null hypotheses are true and all gene expression levels are independent. For ordinary alpha levels (e.g., 0.05), the quantity 1-(1-α)^{k} will be essentially 1 for values of k in the thousands. Alternatively, if a correction is applied, statistical tests may have low power for all but the very largest effects.

The present invention includes a mixture model-based method for the study of microarrays. This method for modeling the distribution of p-values in microarray studies estimates the true positive, true negative, and false discovery rates. In the method, Xᵢ, i=1, . . . ,k represent a P-value from a two-sided statistical test for testing whether there is no difference in expression between two groups for the i^{th} gene. If the genes are independent, then the distribution of Xᵢ is uniform on the interval [0,1] if there is no difference in expression for all genes. If the alternative hypothesis is true for some genes, the distribution may have a peak near zero. Under this setting, we use fact that a distribution on the interval [0,1] can be modeled as a mixture of v beta distributions with parameters rⱼ and sⱼ,j=1 . . . v. The probability density function for Xᵢ is then:

$${f}_{{X}_{i}} ( {x}_{i} ; \underline{λ} , \underline{r} , \underline{s} ) = {λ}_{0} {I}_{( 0 , 1 )} ( {x}_{i} ) + {\sum }_{j = 1}^{v} {λ}_{j} {I}_{( 0 , 1 )} ( {x}_{i} ) \frac{{{x}_{i}^{{r}_{j} - 1} ( 1 - {x}_{i} )}^{{s}_{j} - 1}}{B ( {r}_{j} , {s}_{j} )} ,$$

where B(rⱼ,sⱼ)=∫₀¹u^{r}^{ⱼ}⁻¹(1−u)^{s}^{ⱼ}⁻¹du. The product of this expression over xᵢ, i=1, . . . ,k is called the likelihood when all genes are independent. However, even in the absence of independence, it still provides for a valid index of relative model fit. The number of components and the parameter values in the above expression can be selected using maximum likelihood techniques combined with a bootstrap procedure. This model allows quantification of false positive and false negative probabilities for any particular gene. Simulations have assessed the effect of correlated expression levels on results obtained from the model. Results showed that correlated expression levels do inflate type 1 error rates if left unaccounted for.

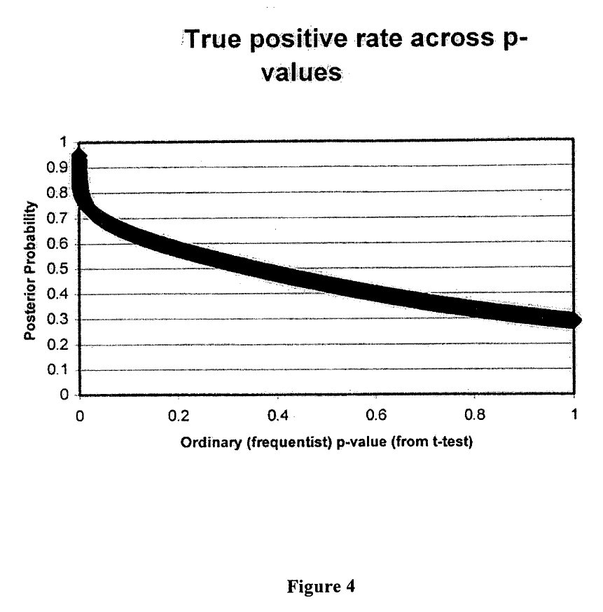

A consequence of the fitted mixture model is the calculation of the posterior probability that a gene was truly differentially expressed as a function of the observed P-value. This is a true positive for a specific gene, denoted TPᵢ, i=1, . . . ,k. For a fitted model that includes a uniform distribution plus one beta distribution component,

$${TP}_{i} = \frac{{λ}_{1} B ( {p}_{i} ; r , s )}{{λ}_{0} {p}_{i} + {λ}_{1} B ( {p}_{i} ; r , s )} ,$$

where B(pᵢ;r,s)is the cumulative distribution function of a beta distribution with shape parameters r and s, evaluated at pᵢ. A plot of TPᵢ versus pᵢ produced the true positive posterior probability plot shown in FIG. 4.

However, certain distributions of P-values might not be appropriate for this technique. The mixture modeling method works for cases when the true positive posterior probability plot is monotonically decreasing, that is, for pᵢ<pⱼ, TPᵢ≧TPⱼ. This implies that the beta distribution is being used to model the smaller P-values in the distribution. Similarly, to produce a true negative posterior probability plot requires computing,

$${TN}_{i} = \frac{{λ}_{0} ( 1 - {p}_{i} )}{{λ}_{0} ( 1 - {p}_{i} ) + {λ}_{1} ( 1 - B ( {p}_{i} ; r , s ) )} .$$

In practice, the posterior probability curves, either true positive or true negative, are actually estimated curves since the parameters of the mixture model were estimated using the data.

The present invention uses a “Alpha Spending Algorithm” to attempt to use the fact that genes may exist in ‘families’ and that members of the same gene family may share similar patterns of gene expression. Consider the following hypothetical example involving the glutathione S-transferase (GST) gene family (McGonigle et al., 2000). An investigator conducts a microarray study comparing the gene expression levels of 1,000 genes for two groups of soybeans that differ with respect to exposure to some xenobiotic stimulus. It is observed that almost all Type III GST gene family members appear to have upregulated expression when exposed to the xenobiotic but the p-values for each gene, though slightly less than 0.05, are far above the level of significance required when adjusting for the large number of genes that have been examined. On the contrary, for the Type II GST family, all genes appear unaffected by the xenobiotic except for one gene with a p-value of 0.001. Ceteris paribus, this smaller p-value is far more compelling. However, many biologists would find the evidence for differential expression of GST Type III genes more compelling. This is because genes that are all members of a common class are responding in a similar fashion. In contrast, because the one Type II gene appears to be acting ‘out of character’ it seems more plausible that the observation is a statistical fluke. Hence, the Alpha Spending algorithm attempts to take the biologist's intuition and translate it into an objective statistical procedure whose stochastic properties can be evaluated.

Moreover, it is not just genes within the same family that may have correlated expression levels, but proteins produced in one gene family often interact with proteins produced by other gene families. In many cases, information about how genes exist in families and how these families interact will be known, or at least presumed known, a priori. Thus, the Alpha Spending algorithm can incorporate, but does not require, a priori biological knowledge.

To apply the Alpha Spending algorithm, one starts with either a priori biological knowledge or a cluster method to subdivide the genes into a number of clusters. For each cluster, a statistical test is applied to detect whether this cluster contains genes that are, on average, differentially expressed. This test is not applied to the individual gene, but rather to the whole cluster as a unit using a bootstrap technique to allow for correlations among genes within clusters. Clusters for which this test is not rejected are discarded while clusters for which this test is rejected are further subdivided and the procedure is repeated. By proceeding in this way the algorithm can end-up with individual genes for which there is significant differential expression. Importantly, a Bonferroni correction need only be applied with respect to the number of clusters rather than the much larger number of genes. The Alpha Spending algorithm is able to perform tests that have far greater power than applying a simple correction for multiple testing, but still hold the overall type 1 (i.e., false positive) error rate to an acceptable level.

Microarray studies frequently measure a large number of variables (genes) on small number of cases. With a sufficiently small number of cases (e.g., N=3 per group), one can neither test whether data are normally distributed nor rely on asymptotic properties of parametric tests, thus, a non-parametric test might be sought. However, with an N as small as 3 per group, conventional non-parametric tests comparing two groups cannot yield 2-tailed p-values <0.10. Thus, it is impossible to obtain “p<0.05,” let alone the far smaller a levels required if one corrected for multiple testing.

The method of the present invention is non-parametric and yet theoretically capable of yielding p-values that are continuous on the interval 0<p≦1 with any N>1 per group. The embodiment discussed here relates to testing for differences between two groups, but the method is generalizable to many other testing situations.

Advantages of the present invention include development of methods to: characterize the reliability and validity of expression measurements; incorporate the information on such measurement properties into experimental designs and inferential procedures; improve the measurement properties of arrays (e.g., via optimal ‘normalization’ procedures); characterize the reliability of a single slide or chip as a quality control check; estimate gene-specific effect sizes using all available information; determine when genes are differentially expressed across experimental conditions even in the face of non-normality and markedly small sample size and to do so through methods that both account for and capitalize on the fact that many genes are examined simultaneously; and develop design and testing strategies involving physical pooling of mRNA and multi-stage testing of effects on gene expression levels that maximize power for and minimize costs.

While many different techniques and methods for the design and analysis of genomic experiments have been described here, it should be understood these individual methods of analysis may be rearranged, separated, deleted, or modified in other ways to according to the needs of the user. Also, it should be understood that the methods disclosed can be readily implemented by various types of computer software. As such, the wide varieties of alternative embodiments of the present invention are intended to be covered as consistent with the language of the claims.

While the invention has been described with respect to a limited number of embodiments, those skilled in the art, having benefit of this disclosure, will appreciate that other embodiments can be devised which do not depart from the scope of the invention as disclosed here. Accordingly, the scope of the invention should be limited only by the attached claims.

What is claimed is:

1. A method for statistical genomic analysis, comprising:
designing a study of a microarray chips of genomic material that utilizes multi-stage testing;
normalizing a measurement of data received from the study;
estimating an experimental error of the measurement of data with an error estimator (θᵢ) for the i^{th} gene is \check{θ}ᵢ=(1−Bᵢ)\hat{θ}ᵢ+Bᵢ\tilde{θ}ᵢ, where Bᵢ=((k−r−2)/ (k−r))Sᵢ/(Sᵢ+Â), where k is the number of genes, where r is the number of predictor variables, and where Sᵢ is the variance of the i^{th} gene; and
inferring conclusions about the expression of the microarray chips of genomic material from the data received from the study based on the calculation of the probability (Xᵢ) that a difference between groups during an experiment happened by chance, where the probability density function for Xᵢ is
$${f}_{{X}_{i}} ( {x}_{i} ; \underline{λ} , \underline{r} , \underline{s} ) = {λ}_{0} {I}_{( 0 , 1 )} ( {x}_{i} ) + {\sum }_{j = 1}^{v} {λ}_{j} {I}_{( 0 , 1 )} ( {x}_{i} ) \frac{{{x}_{i}^{{r}_{j} - 1} ( 1 - {x}_{i} )}^{{s}_{j} - 1}}{B ( {r}_{j} , {s}_{j} )} ,$$
where v is a mixture of beta distributions with parameters rⱼ and sⱼ, j=1 . . . v, and
where B(rⱼ, sⱼ)=, ∫₀¹u^{r}^{ⱼ}⁻¹(1−u)^{s}^{ⱼ}⁻¹du.

2. The method of claim 1, where the microarray chips comprise oligonucleotide chips.

3. The method of claim 1, where the microarray chips comprise cDNA chips.

4. The method of claim 1, where the normalization of a measurement of data occurs within a single microarray chip/slide.

5. The method of claim 1, where the normalization of a measurement of data occurs for a paired microarray chip/slide for dye swap experiments.

6. The method of claim 1, where the normalization of a measurement of data occurs in multiple microarray chip/slides.

7. The method of claim 1, where the normalization of a measurement of data occurs for a same gene on multiple microarray chip/slides.

8. The method of claim 1, where normalizing a measurement of data comprises generating a baseline expression of data.

9. The method of claim 8, where the baseline expression of data is generated for all genes on the microarray chip.

10. The method of claim 8, where the baseline expression of data is generated for housekeeping genes on the microarray chip.

11. The method of claim 8, where the baseline expression of data is generated for control/spikes RNA levels on the microarray chip.

12. The method of claim 1, where the estimation of an experimental error is estimated with an error estimator (EBₖ) is EBₖ=(Rₖ+v)/(Gₖ+v), where R and G are independent samples from gamma distributions with a constant shape parameter, where v=[((a−1)E(R))/c] is a scale parameter assumed to be equal for both R and G, where c is a shape parameter that controls a coefficient of variation (cv) in measurement error, where a controls the cv of actual gene expression, and where E(R) is the overall average intensity in the R sample.

13. The method of claim 1, where the estimation of an experimental error further comprises the computation of a geographic position index of the microarray chip's signal intensity.

14. The method of claim 13, where the geographic position index is computed by regressing intensity measurements on linear or non-linear functions of the microarray chip's Cartesian coordinates.

15. The method of claim 13, where the geographic position index is computed for an array of k genes on the microarray chip by calculating a kXk matrix of Euclidian distances among the genes with respect to physical position on the microarray chip and computing the kXk matrix of Euclidian distances among the genes with respect to expression intensity.

16. The method of claim 1, where inferring conclusions about the expression of the microarray chips of genomic material further comprises pooling a number of individual observations into a number of pooled groups in which the expression of the microarray chips will be measured.

17. The method of claim 1, where the amount of genomic material is increased via a polymerase chain reaction.

18. The method of claim 1, where inferring conclusions about the expression of the microarray chips of genomic material further comprises subdividing genes of the genomic material into clusters based on a prior knowledge of characteristics, then statistically determining whether the clusters contain genes that are differentially expressed.

19. The method of claim 1, where designing a study of a microarray chips of genomic material further comprises, determining a sample size of genomic material needed by the study based on the true positive rate of the data, the true negative rate of the data, and the proportion of genomic material for which a statistically significant difference of expression exists among the sample size.
